(19) United States
(12) Patent Application Publication
(10) Pub. No.: US 2022/0260608 A1
(43) Pub. Date: Aug. 18, 2022

(54) POWER AND BANDWIDTH MANAGEMENT FOR INERTIAL SENSORS

(71) Applicant: Ssam Sports, Inc., Saddle River, NJ (US)

(72) Inventors: Laszlo Kustra, Budapest (HU); Sang J. Kim, Saddle River, NJ (US)

(21) Appl. No.: 17/673,663

(22) Filed: Feb. 16, 2022

Related U.S. Application Data
(60) Provisional application No. 63/200,130, filed on Feb. 16, 2021.

(51) Int. Cl. G01P 13/00; G06F 3/01; G01P 15/02; G01P 15/18; G01P 3/00

(52) U.S. Cl. CPC G01P 13/00; G06F 3/011; G01P 3/00; G01P 15/18; G01P 15/02

(57) ABSTRACT

A processing device initializes an inertial motion capture sensor affixed to a segment of a subject user's body in a detection mode of operation and measures at least one of angular speed or linear acceleration of the segment of the subject user's body using the inertial motion capture sensor in the detection mode of operation. Responsive to the at least one of the angular speed or linear acceleration satisfying a threshold criterion, the processing device switches the inertial motion capture sensor from the detection mode of operation to a capture mode of operation, captures three-dimensional (3D) motion capture data associated with movement of the segment of the subject user's body, and stores the 3D motion capture data in a data store of the inertial motion capture sensor for subsequent retrieval and analysis by a separate computing device.

### RELATED APPLICATIONS

This application claims the benefit of U.S. Provisional Patent Application No. 63/200,130, filed Feb. 16, 2021, the entire contents of which is hereby incorporated by reference herein.

### TECHNICAL FIELD

The present disclosure is generally related to three dimensional (3D) motion capture systems, and is more specifically related to systems and methods for power and bandwidth management for inertial sensors in 3D motion capture systems.

### BACKGROUND

Three dimensional (3D) motion visualization and data is used to analyze human motion in sports and health applications. 3D motion capture systems can utilize inertial sensors to capture useful information of angles, speed, etc. which can be used to identify poor movement for performance or health.

### BRIEF DESCRIPTION OF THE DRAWINGS

The present disclosure is illustrated by way of example, and not by way of limitation, and can be more fully understood with reference to the following detailed description when considered in connection with the figures in which:

FIG. 1 depicts a high-level component diagram of an illustrative system architecture, in accordance with one or more aspects of the present disclosure.

FIG. 2 is a block diagram illustrating an inertial motion capture sensor, in accordance with one or more aspects of the present disclosure.

FIG. 3 is a flow diagram illustrating method of power and bandwidth management for inertial sensors in 3D motion capture systems in accordance with one or more aspects of the present disclosure.

FIG. 4 is a flow diagram illustrating method of specific movement data capture for inertial sensors in 3D motion capture systems in accordance with one or more aspects of the present disclosure.

FIG. 5 is a flow diagram illustrating method of daily activity data capture for inertial sensors in 3D motion capture systems in accordance with one or more aspects of the present disclosure.

FIG. 6 depicts an example computer system which can perform any one or more of the methods described herein, in accordance with one or more aspects of the present disclosure.

### DETAILED DESCRIPTION

Embodiments of systems and methods for power and bandwidth management for inertial sensors are described herein. Inertial sensors are often used in conjunction with 3D motion capture. The inertial sensors typically have an accelerometer, a gyroscope, and a magnetometer (i.e., a compass), among other components. The components work together to capture 3D motion data and provide that data to a computing system for analysis. When a sensor is placed on a person or object to be analyzed, the magnetometer and accelerometer both provide data that is used to understand the orientation of each sensor, relative to one another, in a global reference frame so that the sensor can capture motion of the person or object accurately.

Depending on the use case of the inertial sensors, capturing 3D motion data over the course of a longer period of time, such as several hours or days, poses certain technical challenges. For example, each inertial sensor can be powered by a battery, which has limited life before needing to be replaced or recharged. In addition, each inertial sensor has limited onboard memory, which has a limited storage capacity for captured sensor data. Over the course of multiple hours or days, the battery life can be drained and the storage capacity can be filled, leading to inoperability of the inertial sensors. In addition, if used over an extended period of time, the inertial sensors can capture and store large amounts of 3D motion capture data which will need to be transferred to a separate computing device, for example, for subsequent analysis. Due to limited communication bandwidth, the transfer of large amounts of data can take a significant length of time (e.g., hours) to complete, which may be impractical for the user. Furthermore, the user may wish to view and/or analyze partial results based on the captured data at some point during the day. Such partial results may be generated using online analysis (e.g., an algorithm that's capable of processing the 3D motion capture data as it's streamed from the inertial sensors). The orientation of each inertial sensor also tends to drift around the vertical axis over time if not properly calibrated, leading to inaccurate measurements. Finally, since many motion capture systems utilize multiple inertial sensors concurrently, the internal clocks of the inertial sensors must remain synchronized in order to analyze the movement of a measured body segment with respect to one another. This can be challenging as the internal clocks often drift at varying speeds. The above issues are all exacerbated as the amount of time for which the inertial sensors are operated increases, and should be addressed in order to maintain a function motion capture system that can be used for an extended period of time without interrupting the users training program or daily routine.

In conventional systems, inertial sensors may capture user movements throughout the day (e.g., all movement data) and either store it in the onboard memory of the inertial sensors or stream it to an associated computing device (e.g., a smartphone, tablet computer, laptop computer, etc.). This approach, however, quickly drains the batteries of the inertial sensors and fills up the memory of the inertial sensors, or else requires the inertial sensors to be within wireless range of the computing device. For example, the inertial sensors can connect to the computing device using a number of different technologies including Bluetooth Low Energy (BLE), WiFi, or others.

Aspects of the present disclosure address the above and other considerations by providing intelligent power and bandwidth management for inertial sensors in 3D motion capture systems. The techniques described herein manage the battery, memory, and bandwidth of the inertial sensors, while allowing complete freedom of movement away from the computing device. In one embodiment, the inertial sensors operate in distinct modes to decide when to capture data (e.g., 3D motion capture data), when to store the data, and when to transmit the data to the computing device, while cutting out as much unnecessary data as possible. The specific logic used to make such determinations can be dependent on the use case (e.g., sports analysis vs. daily activity monitoring).

In one embodiment, processing logic in an inertial sensor affixed to a segment of a subject user's body initializes the sensor in a detection mode of operation. The processing logic measures at least one of angular speed or linear acceleration of the segment of the subject user's body using the inertial sensor in the detection mode of operation and determines that the at least one of the angular speed or linear acceleration satisfies a threshold criterion. In response, the processing logic can switch the inertial sensor from the detection mode of operation to a capture mode of operation and begin capturing 3D motion capture data associated with movement of the segment of the subject user's body for a defined length of time. The 3D motion capture data can be stored in a data store for subsequent retrieval and analysis by a separate computing device. Depending on the implementation (or the specific use case), the 3D motion capture data can include highly detailed angular speed and linear acceleration data (or accelerometer output data from which a rough estimate of the linear acceleration can be calculated) for individual movements (e.g., which may be useful in analyzing the mechanics of a specific athletic movement) or less detailed information indicating a change of rotational or linear direction of the body segment (e.g., which may be useful in tracking daily activities, such as step count).

In one embodiment, the computing device can include separate processing logic, such as an activity detection engine, that can analyze the less detailed information to identify a specific activity being performed. For example, the activity detection engine can identify events in the received data (e.g., local maximum and minimum values in the angular movement data) and feed those events to a statistical model (e.g., a Hidden Markov Model (HMM)) which can provide a suspected activity as an output. The suspected activity can be chosen from a preselected set of possible activities, such as walking on level ground, walking up stairs, walking down stairs, standing up, crouching down, etc.

Advantages of the approach described herein include, but are not limited to, improved performance in the 3D motion capture system. By switching between a detection mode and a capture mode, the inertial sensors can limit the 3D motion capture data that is captured and stored to preserve battery life and memory capacity. In addition, since the amount of data captured and stored is reduced, so too is the amount of data transferred from the inertial sensors to a separate communication device. This preserves the limited communication bandwidth between the inertial sensors and the communication device and allows the captured data to be transferred in a reasonable amount of time. Additional details with respect to the power and bandwidth management for inertial sensors are provided below.

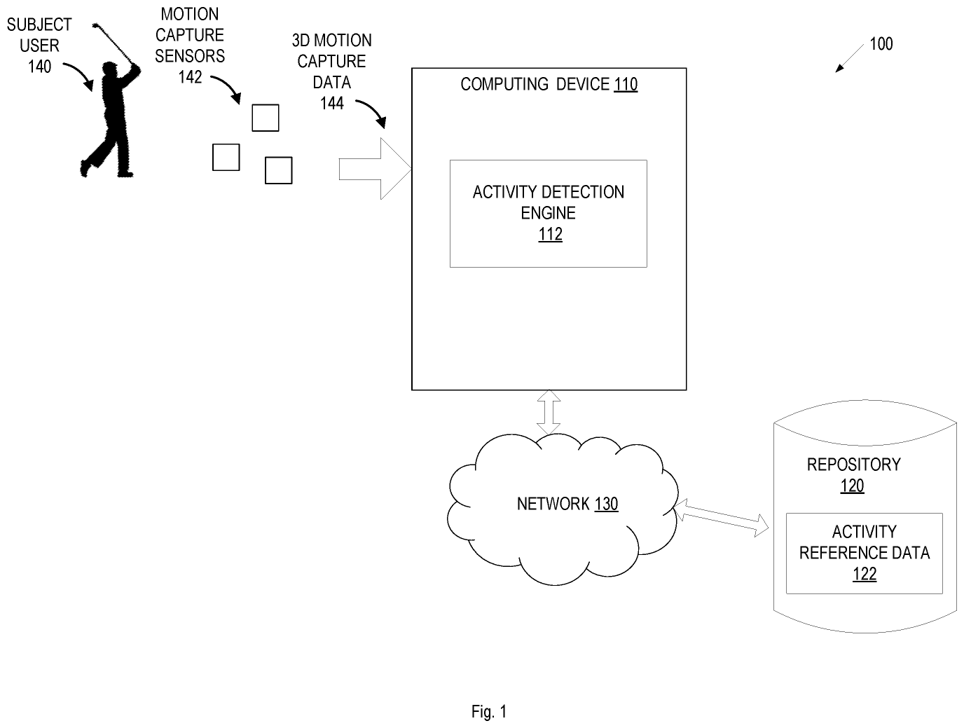

FIG. 1 depicts a high-level component diagram of an illustrative system architecture 100, in accordance with one or more aspects of the present disclosure. System architecture 100 includes a computing device 110 and a repository 120 connected to a network 130. Network 130 may be a public network (e.g., the Internet), a private network (e.g., a local area network (LAN) or wide area network (WAN)), or a combination thereof. In one embodiment, repository 120 can be directly connected to computing device 110 without an intervening network or can be contained within computing device 110.

The computing device 110 may be configured to receive and analyze 3D motion capture data 144 from an array of inertial motion capture sensors 142. In one embodiment, computing device 110 may be a desktop computer, a laptop computer, a smartphone, a tablet computer, a server, or any suitable computing device capable of performing the techniques described herein. In one embodiment, a plurality of motion capture sensors 142, which may be affixed to one or more body parts of a subject user 140 while they are performing a physical activity, capture 3D motion capture data 144 corresponding to the subject user 140. In other embodiments, the motion capture sensors 142 may be affixed to any relevant object being manipulated by the subject user 140 while performing the physical activity, such as to a golf club, baseball bat, tennis racquet, crutches, prosthetics, etc. The 3D motion capture data 144 may be received by the computing device 110.

The 3D motion capture data 144 may be received in any suitable manner. For example, the motion capture sensors 142 may be wireless inertial sensors, each including for example, a gyroscope, magnetometer, accelerometer, and/or other components to measure sensor data including relative positional data, rotational data, and acceleration data. In one embodiment, the motion capture sensors 142 do not include a magnetometer. The 3D motion capture data 144 may include this sensor data and/or other data derived or calculated from the sensor data. The motion capture sensors 142 may transmit the 3D motion capture data 144 including, raw sensor data, filtered sensor data, or calculated sensor data, wirelessly to computing device 110 using internal radios or other communication mechanisms. In other embodiments, other systems may be used to capture 3D motion capture data 144, such as an optical system, using one or more cameras, a mechanical motion system, an electro-magnetic system, an infra-red system, etc. In addition, in other embodiments, the 3D motion capture data 144 may have been previously captured and stored in a database or other data store. In this embodiment, computing device 110 may receive the 3D motion capture data 144 from another computing device or storage device where the 3D motion capture data 144 is maintained. In still other embodiments, the 3D motion capture data 144 may be associated with one or more other users besides or in addition to subject user 140 performing the physical activity.

The 3D motion capture data 144 can be captured by motion capture sensors 142 while the subject user 140 is performing the physical activity. The physical activity can be for example, swinging a golf club, throwing a ball, running, walking, jumping, sitting, standing, or any other physical activity. When performing the physical activity, the subject user 140 may make one or more body movements that together enable performance of the physical activity. For example, when swinging a golf club, the user may rotate their hips and shoulders, swing their arms, hinge their wrists, etc., each of which can be considered a separate body movement associated with performing the physical activity. Each physical activity may have its own unique set of associated body movements.

In one embodiment, each of motion capture sensors 142 includes processing logic that performs power and bandwidth management. In one embodiment, the processing logic causes the respective one of motion capture sensors 142 to operate in distinct modes to decide when to capture data (e.g., 3D motion capture data), when to store the data, and when to transmit the data to the computing device 110. The specific logic used to make such determinations can be dependent on the use case (e.g., sports analysis vs. daily activity monitoring).

In one embodiment, the processing logic in each one of motion capture sensors 142 initializes the sensor in a detection mode of operation. The detection mode is generally a low power mode, where angular speed or linear acceleration are measured, but not stored. The processing logic can determine whether at least one of the angular speed or linear acceleration satisfies a threshold criterion (e.g., meets or exceeds a defined threshold value). In response to the threshold criterion being satisfied, the processing logic can switch the motion capture sensor from the detection mode of operation to a capture mode of operation and begin capturing 3D motion capture data 144 associated with movement of the sensor, and thus the segment of the subject user's body to which the sensor is affixed, for a defined length of time. The 3D motion capture data 144 can be stored in a data store within the respective one of the motion capture sensors 142 for subsequent retrieval and analysis by a computing device 110. Depending on the implementation (or the specific use case), the 3D motion capture data 144 can include highly detailed angular speed and linear acceleration data for individual movements (e.g., which may be useful in analyzing the mechanics of a specific athletic movement) or less detailed information indicating a change of rotational or linear direction of the body segment (e.g., which may be useful in tracking daily activities, such as step count).

In one embodiment, computing device 110 may include activity detection engine 112. The activity detection engine 112 may include instructions stored on one or more tangible, machine-readable storage media of the computing device 110 and executable by one or more processing devices of the computing device 110. In one embodiment, activity detection engine 112 can analyze the 3D motion capture data 144 received from motion capture sensors 142 to identify a specific activity being performed by the subject user 140. For example, the activity detection engine 112 can identify events in the received data (e.g., local maximum and minimum values in the angular movement data) and feed those events to a statistical model (e.g., a Hidden Markov Model (HMM)) which can provide a suspected activity as an output. The suspected activity can be chosen from a preselected set of possible activities, such as walking on level ground, walking up stairs, walking down stairs, standing up, crouching down, etc. For example, the statistical model can compare the sequence of captured events to activity reference data 122 including patterns representing known activities. In one embodiment, 3D motion capture data 144 and activity reference data 122 can be stored in repository 120.

The repository 120 is a persistent storage device that is capable of storing activity reference data 122 and/or other data, as well as data structures to tag, organize, and index this data. Repository 120 may be hosted by one or more storage devices, such as main memory, magnetic or optical storage based disks, tapes or hard drives, NAS, SAN, and so forth. Although depicted as separate from the computing device 110, in an implementation, the repository 120 may be part of the computing device 110 or may be directly attached to computing device 110. In some implementations, repository 120 may be a network-attached file server, while in other embodiments, repository 120 may be some other type of persistent storage such as an object-oriented database, a relational database, and so forth, that may be hosted by a server machine or one or more different machines coupled to the via the network 130.

In one embodiment, the activity detection engine 112 may use a set of trained machine learning models that are trained and used to analyze the 3D motion capture data 144 to identify the physical activity being performed by subject user 140. The physical activity detection engine 112 may also preprocess any received 3D motion capture data, such as 3D motion capture data 144, prior to using the data for training of the set of machine learning models and/or applying the set of trained machine learning models to the data. In some instances, the set of trained machine learning models may be part of the activity detection analysis engine 112 or may be accessed on another machine (e.g., a server machine) by the activity detection engine 112. Based on the output of the set of trained machine learning models, the activity detection engine 112 may obtain an indication of the physical activity being performed.

The server machine on which the trained machine learning models execute may be a rackmount server, a router computer, a personal computer, a portable digital assistant, a mobile phone, a laptop computer, a tablet computer, a camera, a video camera, a netbook, a desktop computer, or any combination of the above. The server machine may include a training engine. The set of machine learning models may refer to model artifacts that are created by the training engine using the training data that includes training inputs and corresponding target outputs (i.e., correct answers for respective training inputs). During training, patterns in the training data that map the training input to the target output (i.e., the answer to be predicted) can be found, and are subsequently used by the machine learning models for future predictions. As described in more detail below, the set of machine learning models may be composed of, e.g., a single level of linear or non-linear operations (e.g., a support vector machine [SVM]) or may be a deep network, i.e., a machine learning model that is composed of multiple levels of non-linear operations). Examples of deep networks are neural networks including convolutional neural networks, recurrent neural networks with one or more hidden layers, and fully connected neural networks. Convolutional neural networks include architectures that may provide efficient physical movement analysis. Convolutional neural networks may include several convolutional layers and subsampling layers that apply filters to portions of the data to detect certain attributes/features. Whereas many machine learning models used for personalized recommendations often suffer from a lack of information about users and their behavior, as well as a lack of relevant input data, activity detection engine 112 has the benefit of high quality information about the users, their physical and demographic attributes, goals and a large amount of movement data. As such, the set of machine learning models, and/or other artificial intelligence models may include, for example, content personalization, collaborative filtering, neural networks or statistical analysis to create high quality movement change recommendations to achieve the desired results. This level of information can allow activity detection engine 112 to make very specific identifications of the physical activity being performed by subject user 140.

As noted above, the set of machine learning models may be trained to determine an activity being performed by subject user 140. Once the set of machine learning models are trained, the set of machine learning models can be provided to activity detection engine 112 for analysis of new 3D motion capture data. For example, activity detection engine 112 may input the new 3D motion capture data into the set of machine learning models. The activity detection engine 112 may then obtain one or more outputs from the set of trained machine learning models.

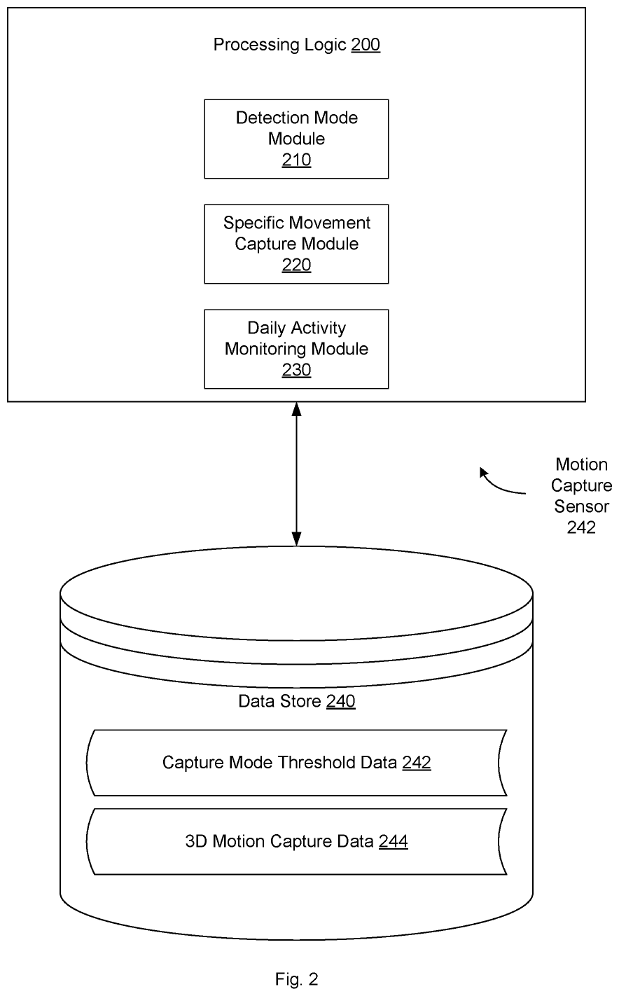

FIG. 2 is a block diagram illustrating an inertial motion capture sensor 242, in accordance with one or more aspects of the present disclosure. In one embodiment, motion capture sensor 242 represents one of motion capture sensors 142, shown in FIG. 1. In one embodiment, motion capture sensor 242 includes processing logic 200, which may include detection mode module 210, specific movement module 220, and daily activity monitoring module 230. This arrangement of modules and components may be a logical separation, and in other embodiments, these modules or other components can be combined together or separated in further components, according to a particular embodiment. In one embodiment, data store 240 is connected to processing logic 200 and includes capture mode threshold data 242 and 3D motion capture data 244. In other embodiments, motion capture sensor 242 may include different and/or additional components which are not shown to simplify the description. Data store 240 may include, for example, a file system, database or other data management layer resident on one or more memory devices or mass storage devices which can include, for example, flash memory; magnetic or optical disks; read-only memory (ROM); random-access memory (RAM); erasable programmable memory (e.g., EPROM and EEPROM); or any other type of storage medium.

In one embodiment, detection mode module 210 initializes the inertial motion capture sensor 242 in a detection mode of operation. In the detection mode, the motion capture sensor 242 consumes less power and doesn't persist the motion capture data in data store 240. Using data from one or more of a gyroscope, magnetometer, accelerometer, and/or other components (not illustrated in FIG. 2), detection mode module 210 measures at least one of angular speed, linear acceleration, or accelerometer output of the motion capture sensor 242, which may be affixed to a segment of the subject user's body, and determines that the at least one of the angular speed or linear acceleration satisfies a threshold criterion. For example, detection mode module 210 can determine that the threshold criterion is satisfied when the measured angular speed, linear acceleration, or accelerometer output meets or exceeds a capture mode threshold, which can be defined and stored as part of capture mode threshold data 242. Motion capture sensor 242 can be configured (e.g., via a mobile application on the computing device) with an angular speed and/or linear acceleration threshold in capture mode threshold data 242 which is appropriate for the specific physical movement and the body segment where the motion capture sensor 242 is to be placed. In another embodiment, motion capture sensor 242 can be configured with an accelerometer output threshold. The accelerometer output contains the effect of gravity, while linear acceleration does not. For example, a stationary sensor's linear acceleration will be zero, while its accelerometer output is around lg, and may be subject to biases that can be rectified by a calibration process. Linear acceleration is a physical measurement and is not subject to the same biases. The accelerometer output will always available on the motion capture sensor 242, as long as the motion capture sensor 242 includes an accelerometer. Linear acceleration can be estimated by a sensor fusion process, an either linear acceleration or accelerometer output can be used for detecting when to capture data (although the accelerometer output will undergo some processing first). For example, the effect of gravity can be mostly removed by a high pass filter, and the result will be good enough for detecting when to capture data, for example, using the threshold criterion. In response to determining that the threshold criterion is satisfied, detection module 210 can notify one of specific movement capture module 220 or daily activity monitoring module 230 to cause the motion capture sensor 242 to switch from the detection mode of operation to a capture mode of operation and begin capturing 3D motion capture data associated with movement of the segment of the subject user's body for a defined length of time.

When performing certain physical movements, such as when playing sports for example, segments of the subject user's body 140 may reach a relatively high angular speed and/or linear acceleration, as measured by motion capture sensor 242. In one embodiment, the physical movement can be a specific and predefined physical movement which is intended to be measured. Such a movement of interest would usually require high frequency capture for proper analysis, and without optimization, this would create too much data to transfer to computing device 110 for processing. For example, for a golf swing, a practical criteria is that within a short time frame, all of the measured motion capture sensors must hit their respective thresholds. With properly set thresholds, this criteria will filter out swings from other, non-swing movements, for example. Conversely, for running, a more optimal criteria may be that when the motion capture sensor placed on the thigh hits its threshold, a capture is triggered for all of the motion capture sensors. In response, specific movement capture module 220 can initiate the capture of 3D motion capture data to monitor the physical movement (e.g., for a defined length of time).

In one embodiment, if the criteria defined in capture mode threshold data 242 is satisfied again while the motion capture sensor 242 is in the capture mode, specific movement capture module 220 can extend the length of the captured data, possibly capturing multiple fast-repeating movements in a single measurement, which will be separated on the computing device 110. This assumes that computing device 110 can tell when there's a gap in the persisted motion capture data 244. One way to do this is to store a timestamp for each frame. Another way is to store the event when the motion capture sensor 242 switches from the capture mode to the detection mode along with the 3D motion capture data 244.

The criteria outlined above triggers a capture well into the physical movement of interest. Accordingly, data for the past few seconds can be continuously maintained in 3D motion capture data 244, and when a capture is triggered, that data can be stored permanently (rather than being deleted after a few seconds). In one embodiment, a few seconds of data after the capture was triggered can also be stored permanently in 3D motion capture data 244. At any other time, data isn't stored permanently in the detection mode, other than always keeping the past few seconds of data to be stored in case a capture is triggered.

This approach uses more battery than being in a completely idle mode, but less battery than writing data to the data store 240 continuously. The largest savings, however, is in data retrieval time, making the limited storage of motion capture sensor 242 practical. The battery usage remains practical for several hours of usage as well.

As described in more detail below, in a system where sensor fusion (e.g., calibration) isn't continuously running on the motion capture sensor 242, but only started as necessary, the battery may last longer. In order to check the detection criteria from capture mode threshold data 242, the raw gyroscope, accelerometer, and/or magnetometer data may be enough, and the sensor's orientation, calculated by sensor fusion, may not be needed (e.g., for angular speed and linear acceleration thresholds). The decision to run or not run sensor fusion on the motion capture sensor 242 is totally independent of the detection and capture modes described above. If the system is designed such that sensor fusion runs on the computing device 110 and not on the motion capture sensor 242, then the criteria for capture mode won't be able to use sensor fusion output. It can still use raw gyroscope, accelerometer, and/or magnetometer data, however, and can persist these when the criteria is met, while running sensor fusion on the computing device 110 on the raw data.

The stored 3D motion capture data 244 can retrieved from the motion capture sensor 242 at any interval of the user's choosing. For example, the user may decide to upload and analyze their data on computing device 110 in the middle of training or at the end of the day. When presenting the data, the system may decide to only keep the time intervals where all of the motion capture sensors were capturing data, or it may present data from each motion capture sensor independently. By using this approach, the motion capture sensor 242 is only capturing the desired physical movements (e.g., golf swing, running, kicking, etc.) while all other movements are not captured and do not need to be sent to the computing device 110. This dramatically reduces battery drain, memory storage requirements, and the amount of data that needs to be sent from the motion capture sensor 242 to the computing device 110.

Alternatively or in addition to the monitoring specific movements by specific movement capture module 220, daily activity monitoring module 230 can track more general movements and daily activities. During everyday activities, people don't usually move with high angular speeds or linear accelerations. Rather smaller and slower movements (e.g. taking a step) are usually performed with high repetition. Users are not interested in looking at each and every movement individually, but may be interested in aggregate metrics (e.g., for jogging, ranges of motion, step counts, step speeds).

In one embodiment, daily activity monitoring module can use the change of direction of the motion capture sensor 242 as a criterion to gather motion data. This information is available from the gyroscope (i.e., change of rotational direction) or the accelerometer (i.e., change of linear direction). In practice, the criterion is satisfied at the beginning and the end of straight line movements, or when the body segment moves on a curved path. For example, while taking a step, two changes of direction occur for both the lower leg and the thigh. While cycling, several changes of direction occur while the user is pedaling.

In one embodiment, at the start of a session, when the user is in a stationary position, motion capture sensor 242 is configured with a threshold for detecting a change of direction. For example, if motion capture sensor 242 is mounted on the pelvis, it may need a different threshold than if it were mounted on the hand with a tremor. In one embodiment, this is an explicit calibration step that involves the user's cooperation. While this step can improve data quality, it isn't strictly necessary and may be omitted in certain embodiments. For example, a threshold for the gyroscope of 45°/s can be used as a default. This threshold can be varied depending on the use case (e.g., for a person with a tremor, or while a person is traveling in a car, etc.).

Daily activity monitoring module 230 can trigger a capture when a change of direction occurs. In practice, this means that a capture may be 10-50 milliseconds long. Depending on the use case, different motion capture sensors may trigger captures independently or in a coordinated manner. When the motion capture sensor 242 is in a stationary position, or when its mid-motion in the same direction, data is not captured. The change of direction data can be stored as 3D motion capture data 244 and can be retrieved from the motion capture sensor 242 by computing device 110. For example, the user may decide to take a look at their data any time during the day, or at the end of the day.

When the 3D motion capture data 244 representing the daily activities and movements of subject user 140 are received by computing device 110, activity detection engine 112 can identify daily living activities, based on the change of direction data from the sensors. This data is in the format of sensor orientations, and optionally linear acceleration.

In one embodiment, activity detection engine 112 first converts orientation data to rotation around the three axes, using a rotation sequence appropriate for the body part. For example, for the thigh and the lower leg, the appropriate rotation sequence may be the ZXZ Euler sequence (in a mediolateral-anteroposterior-longitudinal common reference frame). The first rotation is around the Z (vertical) axis of the common reference frame: azimuth angle. The second rotation is around the new X axis after the first rotation: forward bend angle. The third rotation is around the new Z axis after the second rotation: rotation angle. The resulting rotation angles can have the following properties, for both the thigh and the lower leg. The forward bend angle is 0° when the segment is in attention pose. The forward bend angle is positive when the segment is in front of the body (e.g., flexion for the thigh, extension for the lower leg), and negative when the segment is behind the body (e.g., extension for the thigh, flexion for the lower leg). The rotation angle means the rotation of the segment around its longitudinal axis. The azimuth angle represents the segment's heading in the common reference frame.

As the next step, activity detection engine 112 can find local minimum and maximum values in the forward bend angles for each segment. These local minimum and maximum values (referred to as “events ” herein) are then fed to a Hidden Markov Model, for example. As the general problem being addressed is sequence labeling, other examples that can be used include a Conditional Random Field, Maximum Entropy Markov Model, or some other statistical model.

In one embodiment, different activities have their own state sets, and as events are processed, all activities' states are considered in parallel. Then the state sequence with the maximum likelihood is selected for the received event sequence. The exact state set used depends on the activity itself, and the set of segments measured.

In one embodiment, the Hidden Markov Model includes a number of components. First may be a directed graph, where nodes are the states, and edges are the possible transitions between states. One state always corresponds to exactly one input event. Each edge has an associated transition probability. This isn't a constant number, but can be computed from the past few states while processing incoming events. This probability represents how much a new incoming event can be trusted if there's ambiguity. For example, switching from walking down stairs to cycling is certainly possible, but input events have to be really convincing cycling-like events, otherwise, the model will prefer that the user continued walking down stairs.

Each incoming event can be assigned an observation probability for each possible current state of the system. If the user is mid-step walking down stairs, this indicates the probability of receiving the same event subsequently. As a sequence of input events is received, the activity detection engine 112 doesn't just keep track of a single state. Rather, it keeps track of all possible states and can make a final decision later, when more decisive information is received. These possible states are not equally probable, and there can be a prior probability assigned to each.

After receiving an input event, the next possible states are determined based on these prior probabilities for each current possible state, the transition probabilities, and the observation probabilities. When starting the model, before receiving the first event, each possible starting state is given an initial probability. This will be the “prior” probability when the first event is received. For example, for any type of step detection with the thigh and the lower leg measured, a step detection state subgraph is used. For example, a single step consists of four consecutive events: two events for the thigh and two for the lower leg. A forward state represents an event where the segment is in its most anterior position (i.e. a local maximum value in the forward bend angles). Similarly, a backward state represents a local minimum value in the forward bend angles.

While a sequence of events is received, the model finds the most likely sequence of corresponding states, also called a Viterbi path. Part of this process is assigning a probability to each event and possible source state. The activity detection engine 112 also uses some of the previous states in the partially reconstructed state sequence, as well as the previously received events, especially their timing. Based on the reconstructed sequence of most likely states, presenting activity-related information to the user is trivial, both when data is streamed from the sensors in real time and when all of the data is retrieved from the sensors at the end of the day.

In one embodiment, the probability function described herein determines the probability based on one or more of the following: the ratio of the forward bend angle's curve above the X axis; the curve's shape, especially if has changed compared to the last few steps; the ratio of the ranges of the thigh and forward bend angles during the step.—timing information; and/or the azimuth angle during the step (i.e. did the user make a turn). Some of these items are defined per step type (level ground, downstairs, upstairs). Some of them can be customized per user, based on past data or explicit calibration.

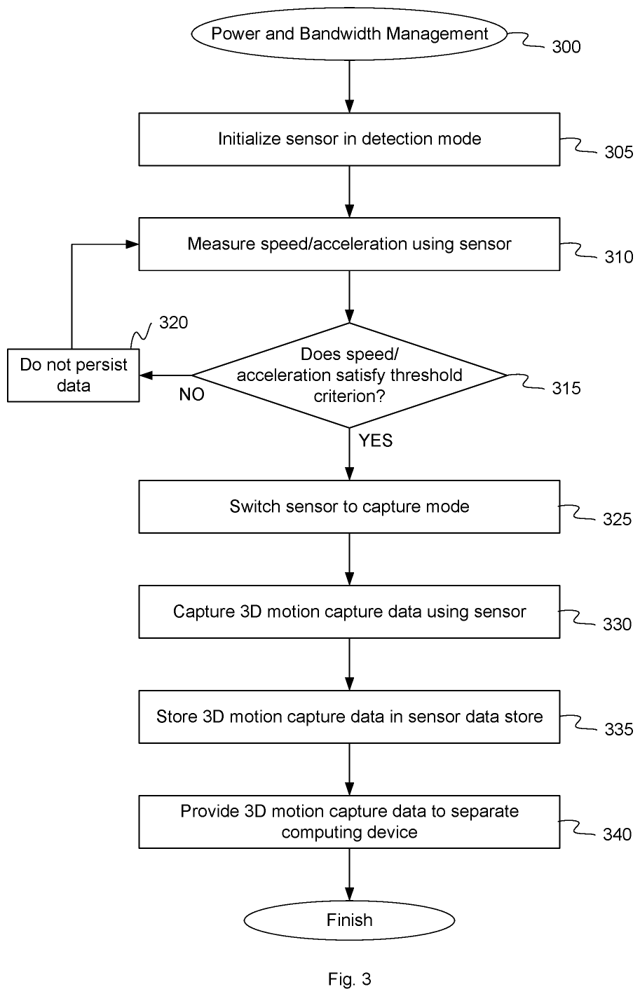

FIG. 3 is a flow diagram illustrating method of power and bandwidth management for inertial sensors in 3D motion capture systems in accordance with one or more aspects of the present disclosure. The method 300 may be performed by processing logic that comprises hardware (e.g., circuitry, dedicated logic, programmable logic, microcode, etc.), software (e.g., instructions run on a processor to perform hardware simulation), firmware, or a combination thereof. In one embodiment, method 300 may be performed by processing logic 200 of motion capture sensor 242, as shown in FIG. 2.

Referring to FIG. 3, at block 305, the processing logic initializes an inertial motion capture sensor, such as motion capture sensor 242 or any of motion capture sensors 142, affixed to a segment of a subject user's body 140 in a detection mode of operation. In the detection mode, the motion capture sensor 242 consumes less power and doesn't persist the motion capture data in data store 240. The processing logic can initialize motion capture sensor 242 in the detection mode automatically upon start-up or re-boot, or in response to a command or request, such as from computing device 110.

At block 310, the processing logic measures at least one of angular speed or linear acceleration of the segment of the subject user's body using the inertial motion capture sensor 242 in the detection mode of operation. Using data from one or more of a gyroscope, magnetometer, accelerometer, and/or other components of the motion capture sensor 242, detection mode module 210 measures at least one of angular speed or linear acceleration of the motion capture sensor 242. As indicated above, this data may be saved only temporarily (e.g., for a few seconds).

At block 315, the processing logic determines whether the at least one of the angular speed or linear acceleration satisfies a threshold criterion. In one embodiment, detection mode module 210 can determine that the threshold criterion is satisfied when the measured angular speed or linear acceleration meets or exceeds a capture mode threshold, which can be defined and stored as part of capture mode threshold data 242. Motion capture sensor 242 can be configured (e.g., via a mobile application on the computing device) with an angular speed and/or linear acceleration threshold in capture mode threshold data 242 which is appropriate for a specific physical movement and the body segment where the motion capture sensor 242 is to be placed.

Responsive to the at least one of the angular speed or linear acceleration not satisfying the threshold criterion, at block 320, the processing logic discards data representing the at least one of the angular speed or linear acceleration.

Responsive to the at least one of the angular speed or linear acceleration satisfying a threshold criterion, at block 325, the processing logic switches the inertial motion capture sensor from the detection mode of operation to a capture mode of operation. In response to determining that the threshold criterion is satisfied, detection module 210 can notify one of specific movement capture module 220 or daily activity monitoring module 230 to cause the motion capture sensor 242 to switch from the detection mode of operation to a capture mode of operation and begin capturing 3D motion capture data associated with movement of the segment of the subject user's body. In another embodiment, the processing logic switches the inertial motion capture sensor 242 from the detection mode of operation to the capture mode of operation responsive to an indication that a different inertial motion capture sensor, such as another one of motion capture sensors 142, satisfies the threshold criterion.

At block 330, the processing logic captures three-dimensional (3D) motion capture data 144 associated with movement of the segment of the subject user's body 140 while the subject user 140 is performing a physical activity. In one embodiment, the motion capture sensors 142 are wireless inertial sensors, each including a gyroscope, magnetometer, accelerometer, and/or other components to measure relative positional data, rotational data, acceleration data, and/or other data. The 3D motion capture data 144 includes data representing one or more body motions associated with performing the physical activity.

At block 335, the processing logic stores the 3D motion capture data in a data store of the inertial motion capture sensor for subsequent retrieval and analysis by a separate computing device. In one embodiment, the data is stored as 3D motion capture data 244 in data store 240 of the motion capture sensor 242.

At block 340, the processing logic providing the stored 3D motion capture data from the inertial motion capture sensor to the separate computing device, such as computing device 110. Depending on the embodiment, the processing logic can provide the 3D motion capture data 244 responsive to a request from the separate computing device 110, responsive to an expiration of a periodic interval (e.g., every few minutes, every hour, etc.), or responsive to some other trigger. For example, if the inertial motion capture sensor is in communication range of computing device 110, the sensor can provide the 3D motion capture data to computing device 110 as soon as the 3D motion capture data is available. If the inertial motion capture sensor is not in communication range of computing device 110 at the time the 3D motion capture data becomes available, then the inertial motion capture sensor can maintain the 3D motion capture data in data store 240 and, once the inertial motion capture sensor is in communication range of computing device 110, the sensor can provide the 3D motion capture data to computing device 110 according to one of the triggers described above.

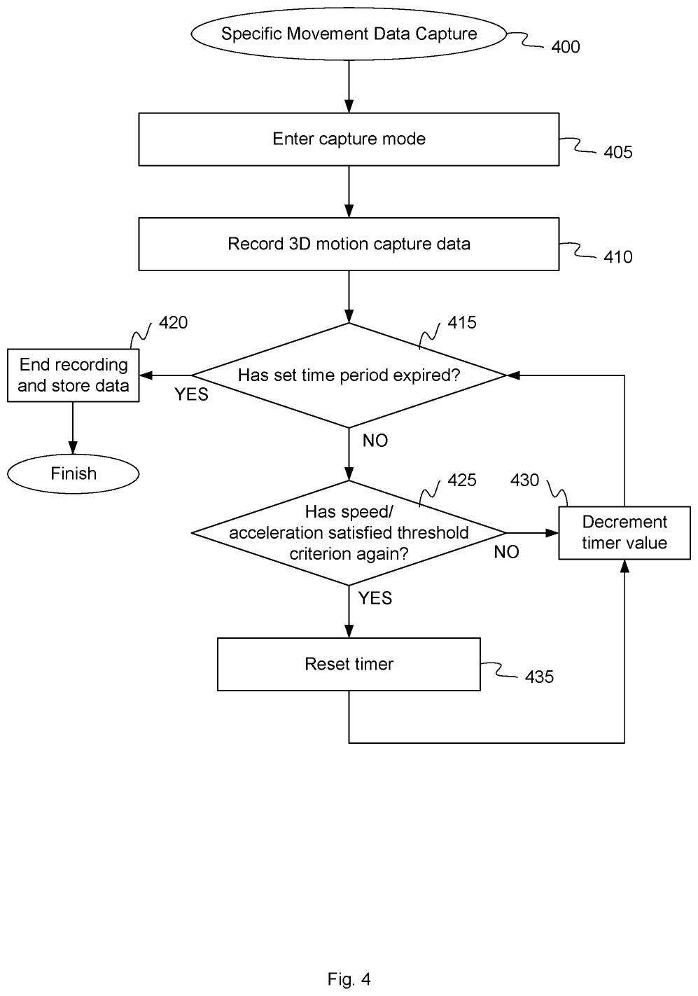

FIG. 4 is a flow diagram illustrating method of specific movement data capture for inertial sensors in 3D motion capture systems in accordance with one or more aspects of the present disclosure. The method 400 may be performed by processing logic that comprises hardware (e.g., circuitry, dedicated logic, programmable logic, microcode, etc.), software (e.g., instructions run on a processor to perform hardware simulation), firmware, or a combination thereof. In one embodiment, method 400 may be performed by processing logic 200 of motion capture sensor 242, as shown in FIG. 2.

Referring to FIG. 4, at block 405, the processing logic causes the inertial motion capture sensor 242 to enter the capture mode of operation. In one embodiment, the capture mode is entered in response to at least one of a measure speed or acceleration of the motion capture sensor 242 satisfying a threshold criterion. In another embodiment, the capture mode is entered responsive to some other trigger, such as a request or command received from a user or from computing device 110.

At block 410, the processing logic records 3D motion capture data 244. As indicated above, the 3D motion capture data 244 can include relative positional data, rotational data, acceleration data, and/or other data measured by one or more of a gyroscope, magnetometer, accelerometer, and/or other components of motion capture sensor 242.

At block 415, the processing logic determines whether a defined length of time (e.g., as indicated by a set time period measured by a timer) has expired. In one embodiment, specific movement capture module 220 includes, or has access to, a timer which is initialized to a default value and then either incremented or decremented until it reaches a final value. When the 3D motion capture data is first recorded at block 410, the timer can be started and recording can continue until the timer expires. If the set time period measured by the timer has expired, at block 420, the processing logic ends the recording and stores the 3D motion capture data 244 in data store 240 of the motion capture sensor.

If the set time period measured by the timer has not expired, however, at block 425, the processing logic determines whether the at least one of the angular speed or linear acceleration has satisfied the threshold criterion again (i.e., whether the measured angular speed or linear acceleration meets or exceeds the capture mode threshold 242 another time after the first time that first triggered the capture mode of operation). If not, at block 430, the processing logic continues to decrement the timer until the set time period has expired. If the angular speed or linear acceleration has satisfied the threshold criterion again, at block 435, the processing logic can reset the timer to the initial value and then proceed to decrement the timer at block 430 until the set time period expires. In this manner, the 3D motion capture data is captured until the specific body movement ends.

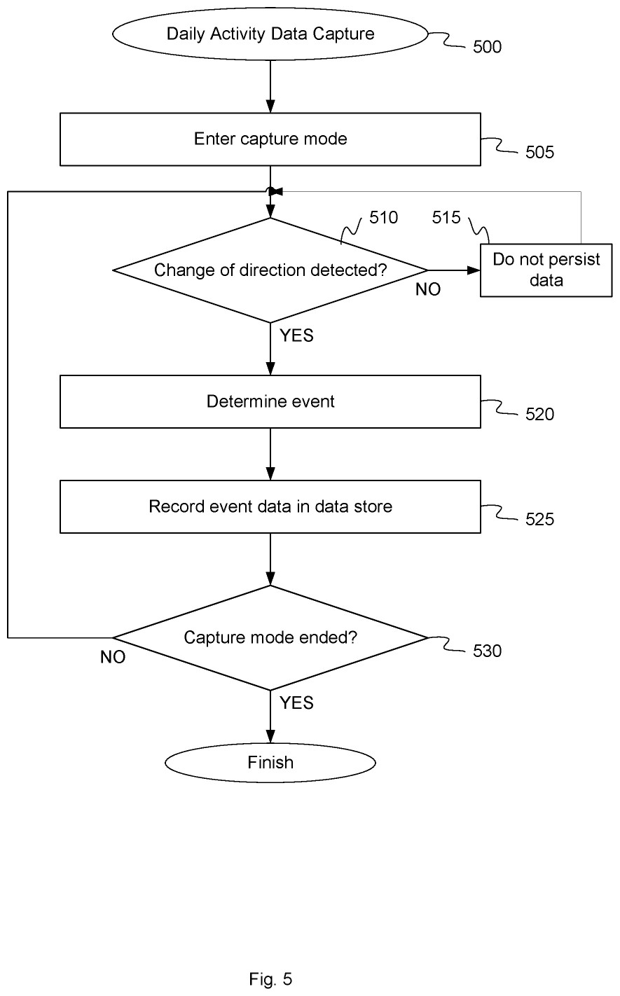

FIG. 5 is a flow diagram illustrating method of daily activity data capture for inertial sensors in 3D motion capture systems in accordance with one or more aspects of the present disclosure. The method 500 may be performed by processing logic that comprises hardware (e.g., circuitry, dedicated logic, programmable logic, microcode, etc.), software (e.g., instructions run on a processor to perform hardware simulation), firmware, or a combination thereof. In one embodiment, method 500 may be performed by processing logic 200 of motion capture sensor 242, as shown in FIG. 2.

Referring to FIG. 5, at block 505, the processing logic causes the inertial motion capture sensor 242 to enter the capture mode of operation. In one embodiment, the capture mode is entered in response to at least one of a measure speed or acceleration of the motion capture sensor 242 satisfying a threshold criterion. In another embodiment, the capture mode is entered responsive to some other trigger, such as a request or command received from a user or from computing device 110.

At block 510, the processing logic determines whether a change of direction is detected in the 3D motion capture data. In one embodiment, daily activity monitoring module 230 uses information from the gyroscope (i.e., change of rotational direction) or the accelerometer (i.e., change of linear direction) to identify the beginning and the end of straight line movements, for example. If no change of direction is detected, at block 515, the processing logic discards the captured data.

If, however, a change of direction is detected, at block 520, the processing logic determines that an event has occurred. Daily activity monitoring module 230 can trigger an event when a change of direction occurs. In practice, this means that a capture associated with the event may be 10-50 milliseconds long. Depending on the use case, different motion capture sensors may trigger captures independently or in a coordinated manner. At block 525, the processing logic records the event data as part of 3D motion capture data 244 in data store 240.

At block 530, the processing logic determines whether the capture mode has ended. Depending on the embodiment, the capture mode is ended after a certain period of time or in response to some other trigger, such as a request or command received from a user or from computing device 110. If the capture mode has not ended, the processing returns to operation 510 and continues the daily activity monitoring operations.

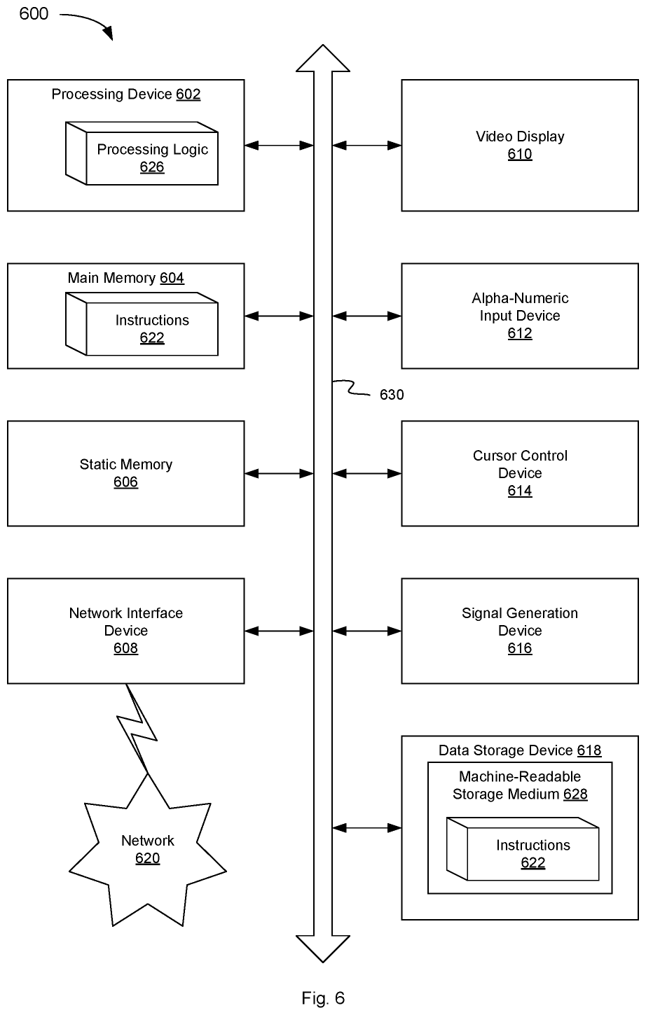

FIG. 6 depicts an example computer system 600 which can perform any one or more of the methods described herein, in accordance with one or more aspects of the present disclosure. In one example, computer system 600 may correspond to a computing device, such as computing device 110, capable of executing activity detection engine 112 of FIG. 1. In another example, computer system 600 may correspond to a motion capture sensor, such as motion capture sensor 242 of FIG. 2, or any of motion capture sensors 142 in FIG. 1. The computer system 600 may be connected (e.g., networked) to other computer systems in a LAN, an intranet, an extranet, or the Internet. The computer system 600 may operate in the capacity of a server in a client-server network environment. The computer system 600 may be a personal computer (PC), a tablet computer, a set-top box (STB), a personal Digital Assistant (PDA), a mobile phone, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The exemplary computer system 600 includes a processing device 602, a main memory 604 (e.g., read-only memory (ROM), flash memory, dynamic random access memory (DRAM) such as synchronous DRAM (SDRAM)), a static memory 606 (e.g., flash memory, static random access memory (SRAM)), and a data storage device 618, which communicate with each other via a bus 630.

Processing device 602 represents one or more general-purpose processing devices such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 602 may also be one or more special-purpose processing devices such as an application specific integrated circuit (ASIC), a field programmable gate array (FPGA), a digital signal processor (DSP), network processor, or the like. The processing device 602 is configured to execute instructions for performing the operations and steps discussed herein.

The computer system 600 may further include a network interface device 608. The computer system 600 also may include a video display unit 610 (e.g., a liquid crystal display (LCD) or a cathode ray tube (CRT)), an alphanumeric input device 612 (e.g., a keyboard), a cursor control device 614 (e.g., a mouse), and a signal generation device 616 (e.g., a speaker). In one illustrative example, the video display unit 610, the alphanumeric input device 612, and the cursor control device 614 may be combined into a single component or device (e.g., an LCD touch screen).

The data storage device 618 may include a computer-readable medium 628 on which the instructions 622 (e.g., implementing activity detection engine 112 or processing logic 200) embodying any one or more of the methodologies or functions described herein is stored. The instructions 622 may also reside, completely or at least partially, within the main memory 604 and/or within the processing device 602 during execution thereof by the computer system 600, the main memory 604 and the processing device 602 also constituting computer-readable media. The instructions 622 may further be transmitted or received over a network via the network interface device 608.

While the computer-readable storage medium 628 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

Although the operations of the methods herein are shown and described in a particular order, the order of the operations of each method may be altered so that certain operations may be performed in an inverse order or so that certain operation may be performed, at least in part, concurrently with other operations. In certain implementations, instructions or sub-operations of distinct operations may be in an intermittent and/or alternating manner.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

In the above description, numerous details are set forth. It will be apparent, however, to one skilled in the art, that the aspects of the present disclosure may be practiced without these specific details. In some instances, well-known structures and devices are shown in block diagram form, rather than in detail, in order to avoid obscuring the present disclosure.

Some portions of the detailed descriptions above are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving,” “determining,” “selecting,” “storing,” “setting,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present disclosure also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear as set forth in the description. In addition, aspects of the present disclosure are not described with reference to any particular programming language. It will be appreciated that a variety of programming languages may be used to implement the teachings of the present disclosure as described herein.

Aspects of the present disclosure may be provided as a computer program product, or software, that may include a machine-readable medium having stored thereon instructions, which may be used to program a computer system (or other electronic devices) to perform a process according to the present disclosure. A machine-readable medium includes any procedure for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.).

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

What is claimed is:

1. A method comprising:
initializing an inertial motion capture sensor affixed to a segment of a subject user's body in a detection mode of operation;
measuring at least one of angular speed or linear acceleration of the segment of the subject user's body using the inertial motion capture sensor in the detection mode of operation;
responsive to the at least one of the angular speed or linear acceleration satisfying a threshold criterion, switching the inertial motion capture sensor from the detection mode of operation to a capture mode of operation;
capturing three-dimensional (3D) motion capture data associated with movement of the segment of the subject user's body; and
storing the 3D motion capture data in a data store of the inertial motion capture sensor for subsequent retrieval and analysis by a separate computing device.

2. The method of claim 1, further comprising:
responsive to capturing and storing the 3D motion capture data, providing the stored 3D motion capture data from the inertial motion capture sensor to the separate computing device when the inertial motion capture sensor is in communication range of the separate computing device.

3. The method of claim 1, further comprising:
providing the stored 3D motion capture data from the inertial motion capture sensor to the separate computing device responsive to at least one of a request from the separate computing device or an expiration of a periodic interval.

4. The method of claim 1, further comprising:
responsive to the at least one of the angular speed or linear acceleration not satisfying the threshold criterion, discarding data representing the at least one of the angular speed or linear acceleration.

5. The method of claim 1, wherein the inertial motion capture sensor is configured to capture specific movement data, and wherein the three-dimensional (3D) motion capture data comprises data associated with the movement of the segment of the subject user's body for a defined length of time.

6. The method of claim 1, wherein the inertial motion capture sensor is configured to capture daily activity data, and wherein the three-dimensional (3D) motion capture data comprises a sequence of events each associated with a change in direction of the movement of the segment of the subject user's body.

7. The method of claim 1, further comprising:
switching the inertial motion capture sensor from the detection mode of operation to the capture mode of operation responsive to an indication that a different inertial motion capture sensor satisfies the threshold criterion.

8. An inertial motion capture sensor comprising:
a data store;
a processing device coupled to the data store, the processing device to perform operations comprising:
initializing the inertial motion capture sensor in a detection mode of operation, wherein the inertial motion capture sensor is affixed to a segment of a subject user's body;
measuring at least one of angular speed or linear acceleration of the segment of the subject user's body using the inertial motion capture sensor in the detection mode of operation;
responsive to the at least one of the angular speed or linear acceleration satisfying a threshold criterion, switching the inertial motion capture sensor from the detection mode of operation to a capture mode of operation;
capturing three-dimensional (3D) motion capture data associated with movement of the segment of the subject user's body; and
storing the 3D motion capture data in the data store for subsequent retrieval and analysis by a separate computing device.

9. The inertial motion capture sensor of claim 8, wherein the processing device is to perform operations further comprising:
responsive to capturing and storing the 3D motion capture data, providing the stored 3D motion capture data from the inertial motion capture sensor to the separate computing device when the inertial motion capture sensor is in communication range of the separate computing device.

10. The inertial motion capture sensor of claim 8, wherein the processing device is to perform operations further comprising:
providing the stored 3D motion capture data from the inertial motion capture sensor to the separate computing device responsive to at least one of a request from the separate computing device or an expiration of a periodic interval.

11. The inertial motion capture sensor of claim 8, wherein the processing device is to perform operations further comprising:
responsive to the at least one of the angular speed or linear acceleration not satisfying the threshold criterion, discarding data representing the at least one of the angular speed or linear acceleration.

12. The inertial motion capture sensor of claim 8, wherein the inertial motion capture sensor is configured to capture specific movement data, and wherein the three-dimensional (3D) motion capture data comprises data associated with the movement of the segment of the subject user's body for a defined length of time.

13. The inertial motion capture sensor of claim 8, wherein the inertial motion capture sensor is configured to capture daily activity data, and wherein the three-dimensional (3D) motion capture data comprises a sequence of events each associated with a change in direction of the movement of the segment of the subject user's body.

14. The inertial motion capture sensor of claim 8, wherein the processing device is to perform operations further comprising:
switching the inertial motion capture sensor from the detection mode of operation to the capture mode of operation responsive to an indication that a different inertial motion capture sensor satisfies the threshold criterion.

15. A non-transitory computer-readable storage medium storing instructions that, when executed by a processing device, cause the processing device to perform operations comprising:
initializing an inertial motion capture sensor affixed to a segment of a subject user's body in a detection mode of operation;
measuring at least one of angular speed or linear acceleration of the segment of the subject user's body using the inertial motion capture sensor in the detection mode of operation;
responsive to the at least one of the angular speed or linear acceleration satisfying a threshold criterion, switching the inertial motion capture sensor from the detection mode of operation to a capture mode of operation;
capturing three-dimensional (3D) motion capture data associated with movement of the segment of the subject user's body; and
storing the 3D motion capture data in a data store of the inertial motion capture sensor for subsequent retrieval and analysis by a separate computing device.

16. The non-transitory computer-readable storage medium of claim 15, wherein the instructions cause the processing device to perform operations further comprising:
responsive to capturing and storing the 3D motion capture data, providing the stored 3D motion capture data from the inertial motion capture sensor to the separate computing device when the inertial motion capture sensor is in communication range of the separate computing device.

17. The non-transitory computer-readable storage medium of claim 15, wherein the instructions cause the processing device to perform operations further comprising:
providing the stored 3D motion capture data from the inertial motion capture sensor to the separate computing device responsive to at least one of a request from the separate computing device or an expiration of a periodic interval.

18. The non-transitory computer-readable storage medium of claim 15, wherein the instructions cause the processing device to perform operations further comprising:
responsive to the at least one of the angular speed or linear acceleration not satisfying the threshold criterion, discarding data representing the at least one of the angular speed or linear acceleration.

19. The non-transitory computer-readable storage medium of claim 15, wherein the inertial motion capture sensor is configured to capture specific movement data, and wherein the three-dimensional (3D) motion capture data comprises data associated with the movement of the segment of the subject user's body for a defined length of time.

20. The non-transitory computer-readable storage medium of claim 15, wherein the inertial motion capture sensor is configured to capture daily activity data, and wherein the three-dimensional (3D) motion capture data comprises a sequence of events each associated with a change in direction of the movement of the segment of the subject user's body.
